(19) United States
(12) Patent Application Publication
(10) Pub. No.: US 2023/0099645 A1
(43) Pub. Date: Mar. 30, 2023

(54) METHOD

(71) Applicant: CEMVITA FACTORY, INC., Houston, TX (US)

(72) Inventors: ZACHARY RICHARD BROUSSARD, Houston, TX (US); Kevin Patrick Kincaid, Houston, TX (US); Mojtaba Karimi, Houston, TX (US); Tahereh Karimi, Houston, TX (US); Mârcio Luis Busi Da Silva, Houston, TX (US); Renata Amanda Gonçalves, Houston, TX (US); Roger Allen Harris, Houston, TX (US); Aaron Christopher Trevino, Houston, TX (US); Luiza Lessa Andrade Lahme, Houston, TX (US); Bárbara De Freitas Magalháes, Houston, TX (US); Christian Walter Rimbau, Houston, TX (US)

(21) Appl. No.: 17/934,765

(22) Filed: Sep. 23, 2022

Related U.S. Application Data
(60) Provisional application No. 63/248,141, filed on Sep. 24, 2021.
(60) Provisional application No. 63/267,568, filed on Feb. 4, 2022.

(51) Int. Cl. C12P 3/00; C01B 32/50; C12P 39/00; B01D 53/22

(52) U.S. Cl. CPC C12P 3/00; C01B 32/50; C12P 39/00; B01D 53/22; C01B 2210/001

(57) ABSTRACT

The present invention provides a process for the microbiological production of hydrogen from a hydrocarbon-rich deposit, said process comprising the step of modifying the composition of the deposit by the introduction into the deposit of at least one non-native hydrogen producing microorganism selected positively to diversify the microbiological abundance of hydrogen-producing microorganisms in the deposit and for the preferential production of hydrogen over methane.

### METHOD

This application claims priority to Provisional Application No. 63/248,141, filed Sep. 24, 2021 and to Provisional Application No. 63/267,568, filed Feb. 4, 2022. The entirety of the aforementioned applications are incorporated herein by reference.

### FIELD

The present invention concerns a process for the microbiological production of hydrogen from a hydrocarbon-rich deposit.

### BACKGROUND

Hydrogen is an important fuel and chemical process substrate. It is known in the art to use microbes to produce hydrogen from hydrocarbon substrates.

WO2005115648 describes a process for characterizing and then manipulating the environment of fermentative syntrophic microorganisms naturally present in a petroleum-bearing subterranean formation in order to promote microbial generation of hydrogen in the formation.

WO2015052806 similarly describes the use of an Fe(III) activator compound to stimulate subterranean microbial hydrogen and methane and also suggests ex situ cultivation and subsequent re-injection of microbes naturally occurring in the subterranean environment.

WO2005113784 describes a method for enhancing microbial production of hydrogen from a hydrocarbon rich deposit. The disclosure favors achieving this by stimulating the metabolic activities of indigenous microorganisms within the deposit, including by the introduction of exogenous (possibly genetically modified) organisms having metabolic capabilities of interest. These metabolic capabilities are not defined except insofar as their impact is to improve net hydrogen production, and contextually this seems to mean by inhibiting the consumption of hydrogen rather than by metabolization of hydrocarbons to hydrogen within the deposit. This document therefore fails to appreciate or to disclose the introduction into the deposit of further microorganisms which are non-native to the deposit and which themselves are capable of metabolizing hydrocarbons to molecular hydrogen and which serve to increase hydrogen production in the deposit by positively diversifying the microbiological abundance of microorganisms in the deposit.

WO0234931 describes a method of generating and recovering methane from solid carbonaceous deposits. This disclosure suggests to inject bacterial consortia into such deposits and recognizes that hydrogen as well as methane may be produced, but methane production is the clear objective of the disclosure and fermentative hydrogen producers are envisioned as being useful only insofar as they provide a feedstock for methanogenesis.

Singh et al., “Overview of Carbon Capture Technology: Microalgal Biorefinery Concept and State-of-the-Art”, Frontiers in Marine Science, 6, 2019, details an overview of carbon capture technology, and in particular microalgal biorefineries, as a means to combat climate change. Singh et al. detail how microalgae can be used to convert raw materials into high and low value products and fuels derived from biomass.

Barnhart et al., “Enhanced coal-dependent methanogenesis coupled with algal biofuels: Potential water recycle and carbon capture”, International Journal of Coal Geology, 171, 2017, 69-75, details methods for stimulating the production of methane from coal bed methanogenesis by introducing further additives to the coal bed to stimulate the activity of the native microorganisms.

Davis', “Organic amendments for enhancing microbial coalbed methane production”, Montana State University, 2017, details the use of organic amendments, i.e. the addition of microbes and/or additives, to enhance the microbial processes for coal-to-methane produced coalbed methane, a form of natural gas found in subsurface coal beds wherein the methane is generated by native microbes to the coal bed. The process detailed therefore focuses on the addition of additives to enhance an already natural process.

In the exemplified prior art examples, the primary focus concerns the manipulation of indigenous microbial populations or their environment, in some cases with the aid of other microbes which inhibit hydrogen consumption or which are themselves methanogenic.

### SUMMARY

According to a first aspect of the present invention there is provided a process for the microbiological production of hydrogen from a hydrocarbon-rich deposit, said process comprising the step of modifying the composition of the deposit by the introduction into the deposit of at least one non-native hydrogen producing microorganism selected positively to diversify the microbiological abundance of hydrogen-producing microorganisms in the deposit and for the preferential production of hydrogen over methane.

The non-native hydrogen producing microorganism may be:

a. a microorganism not naturally present in the hydrocarbon-rich deposit; and/or

b. of a strain of microorganisms not naturally present in the hydrocarbon-rich deposit; and/or

c. of a species of microorganisms not naturally present in the hydrocarbon-rich deposit; and/or

d. of a genus of microorganisms not naturally present in the hydrocarbon-rich deposit; and/or

e. a microorganism naturally present in the hydrocarbon-rich deposit but genetically modified to increase (relative to the naturally present microorganism) its propensity for hydrogen production by the metabolization by that microorganism of one or more hydrocarbons contained within the deposit.

The at least one non-native hydrogen producing microorganism may be one of a plurality of different non-native hydrogen producing microorganisms, strains of microorganisms, species of microorganisms, genera of microorganisms and/or naturally occurring but genetically modified organisms introduced into the deposit. Genetic manipulation of microorganisms naturally present in the deposit to form non-native species may be effected by directed evolution or other form of synthetic biology. The plurality may be greater than two, greater than three, greater then four and/or greater than five.

The non-native hydrogen producing microorganism may have a propensity to metabolize one or more hydrocarbons contained within the deposit to molecular hydrogen in preference to methane such that the yield of production of molecular hydrogen (H2) from the metabolization is higher than the yield of production of methane by at least 1%, by at least 10%, by at least 100% and/or by at least 1000%.

The non-native hydrogen producing microorganism may be introduced into the deposit and accompanied during, after or upon its introduction by at least one nutrient selected to promote the growth of said microorganism and introduced into the deposit for that purpose.

The at least one nutrient may be selected preferentially to promote the growth of the said microorganism in preference to at least one, to at least some or to all of any native microorganisms in the deposit.

The nutrient may comprise one or more of:
a. one or more salts selected from:
   i. phosphates; and/or
   ii. halides; and/or
   iii. nitrates, ammonium salts, nitrogenous salts; and/or
b. one or more carbohydrates selected from:
   i. sugars; and/or
   ii. starches; and/or
c. one or more vitamins; and/or
d. complex nutrients, optionally comprising yeast extracts; corn steep liquor; biomass; bacterial an/or algal biomass.

As will be apparent from Example 11 below it is particularly advantageous to include at least one carbohydrate and/or complex nutrient in the at least one nutrient.

The hydrogen producing microorganism may be introduced into the deposit and accompanied during, after or upon its introduction by at least one pH regulator selected to regulate the pH environment in which the microorganism resides in the deposit and introduced into the deposit for that purpose. The pH regulator may be selected to regulate the pH of the hydrogen producing microorganism environment in the deposit to a pH within the range of from about 5 to about 9, from about 6 to about 8 and/or from about 6 to about 7.

The pH regulator may optionally also serve as a nutrient—for example, phosphate can acts as both a nutrient and as a buffering agent.

The hydrogen producing microorganism may be introduced into the deposit and accompanied during, after or upon its introduction by at least reducing agent which may or may not be included as part of the nutrient package. Suitable reducing agents include thioglycolic acid (and salts such as sodium thioglycolate), cysteine HCl, Na2S, FeS, dithiothreitol, sodium dithionite, ascorbic acid, oxalic acid, sodium sulfite, sodium metabisulfite, 2-mercaptoethanol, sodium pyruvate, glutathione and compatible mixtures of two or more thereof.

The hydrocarbon-rich deposit is preferably a liquid hydrocarbon-rich deposit, e.g oil/bitumen/heavy oil.

The at least one non-native hydrogen producing microorganism may have a genus of Syntrophobacter, Syntrophus, Syntrophomonas, Thermoanaerobacter, Thermotoga, Pseudothermotoga, Thermoanaerobacterium, Fervidobacterium, Thermosipho, Haloanaerobium, Acetoanaerobium, Anaerobaculum, Geotoga, Petrotoga, Thermococcus, Pyrococcus, Clostridium, Enterobacter, Klebsiella, Ethanoligenens, Pantoea, Escherichia, Bacillus, Caldicellulosiruptor, Pelobacter, Caldanaerobacter, Marinitoga, Oceanotoga, Defluviitoga, Kosmotoga, Caloranaerobacter or a combination or mixture thereof.

The at least one non-native hydrogen producing microorganism or the at least one recombinant microorganism may be the same or different.

The non-native hydrogen producing microorganism or the recombinant microorganism may express at least one protein selected from hydrogenases, dehydrogenases, hydroxylases, carboxylases, esterases, hydratases and acetyltransferases having an amino acid sequence at least 95% identical to a sequence expressed by an upregulated or downregulated gene selected from mth (EC 1.12.98.2), mrt, hycA (ID: 45797123), fdhF (ID: 66346687), fhlA (ID: 947181), ldhA (ID: 946315), nuoB (ID: 65303631), hybO (ID: 945902), fdhl, narP, ppk or Pepc by expressing a non-native protein expressing nucleotide sequence, wherein an amount of hydrogen produced or protein produced by the non-native hydrogen producing microorganism or the recombinant microorganism is greater than that produced relative to a control microorganism lacking the non-native protein expressing nucleotide sequence.

The recombinant microorganism may express at least one Coenzyme M reductase and or dehydrogenase protein having a gene sequences at least 95% identical to SEQ ID NO. [mmg:MTBMA_c15480], [mth:MTH_1015], [mmg:MTBMA_c15520], [mmg:MTBMA_c15490], [mth:MTH_1166], [mth:MTH_1167], [eco:b4346], [eco:b4345], [ag:AAA22593], [mea:Mex_1p4538, [mea:Mex_1p4535], [ag:ACS29499], [ag: CAH55641], [mrd:Mrad2831_0508], by expressing a non-native Coenzyme M reductase and or dehydrogenase expressing nucleotide sequence.

Preferably, an amount of hydrogen produced or protein produced by the non-native hydrogen producing microorganism and/or the recombinant microorganism is greater than that produced relative to a control microorganism lacking the non-native protein expressing nucleotide sequence.

The environment of the hydrocarbon-rich deposit and the introduced hydrogen producing microorganism may constitute an enclosed bioreactor, being a bioreactor subterranean formation, a bioreactor landfill enclosure, or a combination thereof.

In this case there is provided in accordance with the aforesaid first aspect of the invention and any or each of its described variants a method of increasing hydrogen production from an enclosed bioreactor (as constituted by the environment of the hydrocarbon-rich deposit and the introduced hydrogen producing microorganism) comprising: providing a baseline reaction mixture in the enclosed bioreactor, wherein the baseline reaction mixture includes a hydrocarbon having up to 120 carbon atoms, water, and a baseline amount of at least one microorganism; producing baseline microorganism data on an identity and a baseline percentage of the at least one microorganism, relative to a baseline total percentage of microorganisms in the baseline reaction mixture, by performing DNA and/or RNA sequencing of a baseline microorganism sample from the baseline reaction mixture; measuring a baseline amount of hydrogen in a baseline gas sample of gasses collected from the enclosed bioreactor; increasing hydrogen production from the enclosed bioreactor by forming a synthetic reaction mixture, and harvesting the hydrogen from the enclosed bioreactor at a hydrogen harvesting rate by separating the hydrogen from other gasses and transferring the hydrogen into a hydrogen storage container.

According to a second aspect of the present invention, there is provided a method of increasing hydrogen production from an enclosed bioreactor comprising: providing at least one anode and at least one cathode connected to an interior of the enclosed bioreactor, wherein the enclosed bioreactor is a subterranean formation, an enclosed landfill, or a combination thereof, and the at least one anode and the at least one cathode are connected through the enclosed bioreactor by at least one bioreactor liquid pathway; providing a baseline reaction mixture in the enclosed bioreactor, wherein the baseline reaction mixture includes an organic substrate, water, and a baseline amount of at least one microorganism; measuring a baseline amount of hydrogen in a baseline gas sample of gasses collected from the enclosed bioreactor; increasing hydrogen production from the enclosed bioreactor from the baseline amount of hydrogen to a production amount of hydrogen by applying a potential between the at least one anode and the at least one cathode; and harvesting the hydrogen from the enclosed bioreactor at a hydrogen harvesting rate by separating the hydrogen from other gasses and transferring the hydrogen into a hydrogen storage container, wherein the production amount of hydrogen is at least 20% greater than the baseline amount of hydrogen.

The inventors of the present invention have surprisingly found that by introducing an anode and cathode to the bioreactor, the microbes can be encouraged to produce more hydrogen. This is particularly beneficial at times where electricity is cheap and in plentiful supply. For example, this cheap electricity could be used to convert and store a greater amount of hydrogen for use when electricity is more expensive.

The at least one cathode may include two or more cathodes and/or the at least one anode may include two or more anodes connected to the enclosed bioreactor. The at least one anode, the at least one cathode, or a combination thereof may include at least one wellbore casing electrically connected to a power source.

A closest distance between an anode of the at least one anode and a cathode of the at least one cathode may be from 100 m to 1000 m.

The at least one anode and the at least one cathode may be electrically connected to a at least one power source. The at least one power source may include a wind turbine, a solar cell, an electric dam, a power grid, or a combination thereof. The at least one anode and the at least one cathode may be directly electrically connected to a at least one power source. The at least one power source may include a wind turbine, a solar cell, or a combination thereof.

The method may comprise applying a potential between the at least one anode and the at least one cathode of from about 0.6 V to about 9.0 V. The method may comprise applying a voltage per cubic meter in the enclosed bioreactor of from about 0.1 V/m3 to a about 0.5 V/m3 as measured a distance of from about 10 m to about 50 m from the at least one anode or the at least one cathode.

According to a third aspect of the present invention, there is provided a method of increasing production of a hydrogen-containing liquid from an enclosed bioreactor comprising: providing a baseline reaction mixture in the enclosed bioreactor, wherein the baseline reaction mixture includes a substrate, water, and a baseline amount of at least one microorganism, wherein the substrate includes a nitrogen source, an unsaturated hydrocarbon having from 2 to 120 carbon atoms, methane, hydrogen, or a combination thereof, wherein the hydrogen-containing liquid includes ammonia, ammonium, methanol, a saturated hydrocarbon having from 2 to 120 carbon atoms, or a combination thereof producing baseline microorganism data on an identity and a baseline percentage of the at least one microorganism, relative to a baseline total percentage of microorganisms in the baseline reaction mixture, by performing DNA and/or RNA sequencing of a baseline microorganism sample from the baseline reaction mixture; measuring a baseline amount of hydrogen-containing liquid in a baseline sample collected from the enclosed bioreactor; increasing production of the hydrogen-containing liquid from the enclosed bioreactor by forming a synthetic reaction mixture, and harvesting the hydrogen-containing liquid from the enclosed bioreactor at a hydrogen-containing liquid harvesting rate by separating the hydrogen-containing liquid from solids and other liquids by transferring the hydrogen-containing liquid into a hydrogen-containing liquid storage container.

The nitrogen source may include nitrogen gas, agriculture waste, soy protein isolate, blood meal, feather meal, dried fish, yeast extract, nitrates, nitrites, urea, soy flour, peanut cake, peptone, beef extract, or a combination thereof.

According to a fourth aspect of the present invention, there is provided a method of hydrogen production and hydrocarbon wastewater purification comprising: providing the hydrocarbon wastewater from a hydrocarbon producing site; forming a baseline reaction mixture by transferring the hydrocarbon wastewater into an enclosed bioreactor, wherein the baseline reaction mixture includes the hydrocarbon wastewater and a baseline amount of at least one microorganism; producing baseline microorganism data on an identity and a baseline percentage of the at least one microorganism, relative to a baseline total percentage of microorganisms in the baseline reaction mixture, by performing DNA and/or RNA sequencing of a baseline microorganism sample from the baseline reaction mixture; measuring a baseline amount of hydrogen in a baseline gas sample of gasses collected from the enclosed bioreactor; measuring a baseline amount of hydrocarbons in a baseline liquid sample of a liquid collected from the enclosed bioreactor; producing hydrogen and forming purified water from the hydrocarbon wastewater by forming a synthetic reaction mixture in the enclosed bioreactor, harvesting the hydrogen from the enclosed bioreactor at a hydrogen harvesting rate by separating the hydrogen from other gasses and transferring the hydrogen into a hydrogen storage container, and gathering the purified water from the enclosed bioreactor by transferring the purified water from the enclosed bioreactor to a purified water liquid path at a purified water rate, for example of from about 10 L/hr to about 10,000 L/hr.

The synthetic reaction mixture is formed by: adding at least one non-native hydrogen producing microorganism until a percentage of the non-native hydrogen producing microorganism in the synthetic reaction mixture is at least 20% of a total amount of microorganisms in the synthetic reaction mixture; or adding at least one hydrogen production enhancer to the baseline reaction mixture until a post-baseline amount of hydrogen in a post-baseline gas sample of gasses collected from the enclosed bioreactor is at least 10% higher than the baseline amount of hydrogen; or adding at least one recombinant microorganism to the baseline reaction mixture until a percentage of the at least one recombinant microorganism in the synthetic reaction mixture is at least 20% of a total amount of microorganisms in the reaction mixture, or a combination thereof. The enclosed bioreactor is a bioreactor subterranean formation, a bioreactor landfill enclosure, or a combination thereof.

The method may further comprise after providing the baseline reaction mixture, but before forming the synthetic reaction mixture, producing baseline environmental data from the baseline reaction mixture. The baseline environmental data may include one or more of the following measurements of a baseline environmental sample from the baseline reaction mixture: pH; temperature; water analysis; oxidation-reduction potential; pressure; dissolved oxygen; hydrocarbon concentrations; volatile fatty acids concentrations; cation concentration; anion concentration; concentration of gases (such as one or more of NH₃, CO₂, CO, H₂, H₂S and CH₄); salt concentration; and metal concentration.

The baseline microorganism sample and the baseline environmental sample may be the same or different.

The hydrogen production rate may be at least about 0.1 L/hr, or at least about 1 L/hr, or at least about 10 L/hr, or at least about 100 L/hr. The hydrogen production rate may be up to about 106 L/hr, or up to about 105 L/hr, or up to about 104 L/hr, or up to about 103 L/hr. The hydrogen production rate may be from about 0.1 L/hr to about 106 L/hr, or from about 0.1 L/hr to about 103 L/hr, or from about 103 L/hr to about 106 L/hr.

The organic mass may include a hydrocarbon having up to 120 carbon atoms, or from 1-70 carbon atoms, or from 1 to 40 carbon atoms, or from 1 to 4 carbon atoms, a biodegradable waste, a paper waste, a plant waste, a pulp waste, or a combination thereof. The unsaturated hydrocarbon having from 2 to about 120 carbon atoms may include an alkene, an alkyne, an aromatic hydrocarbon, or a polyaromatic hydrocarbon.

The subterranean formation may include a natural formation, non-natural formation, a hydrocarbon-bearing formation, a natural gas-bearing formation, a methane-bearing formation, a depleted hydrocarbon formation, a depleted natural gas-bearing formation, a wellbore, or a combination thereof.

The bioreactor landfill enclosure may include a landfill that is enclosed by a building material. The building material may include at least one of a brick, a cement, a plastic, a non-natural rubber, a geomembrane of any kind, concrete, steel, a glass, or a combination thereof.

The at least one bioreactor liquid pathway may be a natural subterranean formation, a constructed subterranean opening, a drilled opening, or one or more gaps between waste in a landfill, or a combination thereof.

The hydrogen production enhancer may be a biocidal inhibitor, a methanogenesis inhibitor, a sulfate reduction inhibitor, a nitrate reduction inhibitor, an iron reduction inhibitor, or a combination thereof.

The biocidal inhibitor may be glutaraldehyde, a quaternary ammonium compound, formaldehyde, a formaldehyde releaser such as 3,3′-methylenebis[5-methyloxazolidine], dibromonitrilopropionamide, tetrakis hydroxymethyl phosphonium sulfate, chlorine dioxide, peracetic acid, tributyl tetradecyl phosphonium chloride, methylisothiazolinone, chloromethylisothiazolinone, sodium hypochlorite, dazomet, dimethyloxazolidine, trimethyloxazolidine, N-bromosuccinimide, bronopol, or 2-propenal, or a mixture thereof.

The methanogenesis inhibitor may be bromethane sulfonic acid, an aminobenzoic acid, 2-bromoethanesulfonate, 2-chloroethanesulfonate, 2-mercaptoethanesulfonate, lumazine, a fluoroacetate, nitroethane, or 2-nitropropanol, or a mixture thereof.

The sulfate reduction inhibitor may be a molybdate salt, a nitrate salt, a nitrite salt, a chlorate salt, or a perchlorate salt or a mixture thereof.

The nitrate reduction inhibitor may be sodium chlorate, a chlorate salt, or a perchlorate salt, or a mixture thereof.

The method may further comprise producing carbon dioxide from the enclosed bioreactor at a carbon dioxide producing rate, and separating the carbon dioxide from other gasses by filtering the carbon dioxide through a carbon dioxide-selective membrane filter; and pumping the carbon dioxide into the enclosed bioreactor at a replenishment rate or to a different enclosed bioreactor at an injection rate; or forming an algal (phototrophic) biomass by reacting the carbon dioxide with an algae (phototrophic) reaction mixture in an algal (phototrophic) bioreactor, and pumping the algal (phototrophic) biomass into the reaction mixture of the enclosed bioreactor or a different enclosed bioreactor.

The method may further comprise harvesting hydrogen from the enclosed bioreactor at a hydrogen harvesting rate, and separating the hydrogen from other gasses by filtering the hydrogen through a hydrogen-selective membrane filter and transferring the hydrogen into a hydrogen storage container.

The method may further comprise harvesting the hydrogen from the enclosed bioreactor by accessing a resealable hydrogen gas path located closer to the at a least one cathode than any anode of the at least one anode.

The method may further comprise harvesting the carbon dioxide from the enclosed bioreactor by accessing a resealable carbon dioxide gas path located closer to the at a least one anode than any cathode of the at least one cathode.

Forming the synthetic reaction mixture may include one or more of:

a. adding at least one non-native hydrogen producing microorganism until a percentage of the non-native hydrogen producing microorganism in the synthetic reaction mixture is at least 20% of a total amount of microorganisms in the synthetic reaction mixture;

b. adding at least one hydrogen production enhancer to the baseline reaction mixture until a post-baseline amount of hydrogen in a post-baseline gas sample of gasses collected from the enclosed bioreactor is at least 10% higher than the baseline amount of hydrogen; and/or

c. adding at least one recombinant microorganism to the baseline reaction mixture until a percentage of the at least one recombinant microorganism in the synthetic reaction mixture is at least 20% of a total amount of microorganisms in the reaction mixture; and/or

d. adding at least one electro-synthetic microorganism to the baseline reaction mixture until a percentage of the at least one recombinant microorganism in the synthetic reaction mixture is at least 20% of a total amount of microorganisms in the reaction mixture.

The method may further comprise detecting at least one residual hydrocarbon in the purified water in the purified water liquid path.

The method may further comprise the steps of:

a. forming a second baseline reaction mixture by transferring the purified water into a second enclosed bioreactor, wherein the second baseline reaction mixture includes a second baseline amount of at least one microorganism and the purified water, wherein the purified water contains the at least one residual hydrocarbon;

b. producing second baseline microorganism data on a second identity and a second baseline percentage of the at least one microorganism, relative to a second baseline total percentage of microorganisms in the second baseline reaction mixture, by performing DNA and/or RNA sequencing of a second baseline microorganism sample from the second baseline reaction mixture;

c. measuring a second baseline amount of hydrogen in a second baseline gas sample of gasses collected from the second enclosed bioreactor;

d. measuring a second baseline amount of hydrocarbons in a second baseline liquid sample of a second liquid collected from the second enclosed bioreactor;

e. producing hydrogen and forming a second purified water from the purified water by forming a second synthetic reaction mixture in the second enclosed bioreactor;

f. harvesting the hydrogen from the second enclosed bioreactor at a second hydrogen harvesting rate by separating the hydrogen from other gasses and transferring the hydrogen into a second hydrogen storage container; and

g. gathering the second purified water from the enclosed bioreactor by transferring the second purified water from the second enclosed bioreactor to a second purified water path at a second purified water rate.

The second synthetic reaction mixture may be formed by one or more of:

a. adding at least one second non-native hydrogen producing microorganism until a second percentage of the second non-native hydrogen producing microorganism in the second synthetic reaction mixture is at least 20% of a second total amount of microorganisms in the second synthetic reaction mixture;

b. adding at least one second hydrogen production enhancer to the second baseline reaction mixture until a second post-baseline amount of hydrogen in a second post-baseline gas sample of gasses collected from the second enclosed bioreactor is at least 10% higher than the second baseline amount of hydrogen;

c. adding at least one second recombinant microorganism to the second baseline reaction mixture until a percentage of the at least one second recombinant microorganism in the second synthetic reaction mixture is at least 20% of a second total amount of microorganisms in the second synthetic reaction mixture, and/or

d. a combination thereof.

The second enclosed bioreactor may be a second lined surface formation, a second lined pool, or a combination thereof.

According to a fifth aspect of the present invention, there is provided a system for increasing hydrogen production from an enclosed bioreactor comprising: an enclosed bioreactor, a hydrogen storage container, a hydrogen separator, and an algal bioreactor. The enclosed bioreactor contains a reaction mixture, wherein the reaction mixture includes methane, water, a biomass, and a production amount of at least one microorganism. The hydrogen separator includes at least one hydrogen-selective membrane filter. The algal (or phototrophic organism) bioreactor contains a carbon dioxide, oxygen, and an algae reaction mixture. The algae reaction mixture includes water and at least one alga. The enclosed bioreactor is connected to the hydrogen separator by a hydrogen gas path. The algal bioreactor is connected to by a carbon dioxide gas path to the hydrogen separator or the enclosed bioreactor. The algal bioreactor is connected to the enclosed bioreactor by a biomass gas path or a biomass liquid path or a combination thereof. The hydrogen separator is connected to the hydrogen storage container by a filtered hydrogen gas path.

The enclosed bioreactor may have a volume of at least about 100 m3, or at least about 103 m3, or at least about 104 m3, or at least about 105 m3. The enclosed bioreactor may have a volume of up to about 4×109 m3, or up to about 4×108 m3, or up to about 4×107 m3, or up to about 4×106 m3. The enclosed bioreactor may have a volume of from about 100 m3 to about 4×109 m3, or from about 100 m3 to about 4×106 m3, or from about 4×106 m3 to about 4×109 m3.

The algal bioreactor may have a volume of from about 100 m3 to about 2,000 m3.

The enclosed bioreactor may include a bioreactor subterranean formation or a bioreactor landfill enclosure.

The bioreactor subterranean formation may include a natural formation, non-natural formation, a hydrocarbon-bearing formation, a natural gas-bearing formation, a methane-bearing formation, a depleted hydrocarbon formation, a depleted natural gas-bearing formation, a wellbore, or a combination thereof.

The bioreactor landfill enclosure may include a landfill that is enclosed by a building material. The building material may include at least one of a brick, a cement, a plastic, a non-natural rubber, a geomembrane of any kind, concrete, steel, a glass, or a combination thereof.

The hydrogen storage container may be a gas tank, a hydrogen subterranean formation, or a hydrogen artificial enclosure.

The hydrogen subterranean formation may include a natural formation or non-natural formation.

The hydrogen artificial enclosure may be made of one or more building materials. The building materials may include a cement, a plastic, a non-natural rubber, a geomembrane of any kind, concrete, a metal or metal alloy (such as steel), or a combination thereof.

The system may further comprise a genetic material testing facility, preferably within about 1000 meters of a resealable opening of the enclosed bioreactor. The genetic material testing facility may contain at least one DNA and/or RNA sequencer.

The at least one cathode may include two or more cathodes and/or the at least one anode may include two or more anodes connected to the enclosed bioreactor.

The at least one anode, the at least one cathode, or a combination thereof may include a wellbore casing electrically connected to a power source.

A closest distance between an anode of the at least one anode and a cathode of the at least one cathode may be from 100 m to 1000 m.

The at least one anode and the at least one cathode may be electrically connected to a at least one power source. The at least one power source may include a wind turbine, a solar cell, an electric dam, a power grid, or a combination thereof.

The enclosed bioreactor may further include a resealable hydrogen gas path located closer to the at a least one cathode than any anode of the at least one anode and the resealable hydrogen gas path connects to the interior of the enclosed bioreactor.

The enclosed bioreactor may further include a resealable carbon dioxide gas path located closer to the at a least one anode than any cathode of the at least one cathode and the resealable carbon dioxide gas path connects to the interior of the enclosed bioreactor.

The system may further comprise at least one microorganism container or at least one hydrogen production enhancer container or a combination thereof.

The at least one microorganism container and/or the at least one hydrogen production enhancer container may be connected to the enclosed bioreactor by an additive solid pathway or an additive liquid pathway.

According to a sixth aspect of the present invention, there is provided a system for increasing hydrogen production and hydrocarbon wastewater purification comprising an enclosed bioreactor, a hydrogen separator, a hydrogen storage container, a purified water liquid path, and a hydrocarbon wastewater intake. The enclosed bioreactor contains a reaction mixture. The reaction mixture includes a hydrocarbon wastewater and a baseline amount of at least one microorganism. The hydrocarbon wastewater intake is a wastewater liquid path connects the enclosed bioreactor to a hydrocarbon producing site or a hydrocarbon wastewater receptacle. The enclosed bioreactor is connected to the hydrogen separator by a hydrogen gas path. The hydrogen separator is connected to the hydrogen storage container by a filtered hydrogen gas path. The purified water liquid path is connected to the enclosed bioreactor. The enclosed bioreactor is a lined surface formation, a lined pool, or a combination thereof.

A bottom of the enclosed bioreactor may be lined with a hydrocarbon impermeable material.

The enclosed bioreactor may be covered by a hydrogen impermeable material.

The enclosed bioreactor may have a volume of at least about 100 m3, or at least about 103 m3, or at least about 104 m3, or at least about 105 m3. The enclosed bioreactor may have a volume of up to about 4×109 m3, or up to about 4×108 m3, or up to about 4×107 m3, or up to about 4×106 m3. The enclosed bioreactor may have a volume of from about 100 m3 to about 4×109 m3, or from about 100 m3 to about 4×106 m3, or from about 4×106 m3 to about 4×109 m3.

The lined surface formation may include a natural formation or non-natural formation.

The hydrogen artificial enclosure may be made of one or more building materials. The building materials may include a cement, a plastic, a non-natural rubber, a geomembrane of any kind, concrete, a metal or a metal alloy (such as steel), or a combination thereof.

The system may further comprise a genetic material testing facility, preferably within about 1000 meters of a resealable opening of the enclosed bioreactor and connected to the resealable opening by a genetic material testing liquid pathway. The genetic material testing facility may contain at least one DNA and/or RNA sequencer.

The system may further comprise at least one microorganism container or at least one hydrogen production enhancer container or a combination thereof.

The at least one microorganism container and/or the at least one hydrogen production enhancer container may be connected to the enclosed bioreactor by an additive solid pathway or an additive liquid pathway.

The purified water liquid path may contain at least one hydrocarbon sensor or at least one resealable sample port.

The purified water liquid path may connect to a second enclosed bioreactor containing a second reaction mixture. The second reaction mixture may include a purified water, wherein the purified water contains the at least one residual hydrocarbon.

The second enclosed bioreactor may be connected to a second hydrogen separator by a second hydrogen gas path.

A second hydrogen separator may be connected to a second hydrogen storage container by a second filtered hydrogen gas path.

The hydrogen separator and the second hydrogen separator may be the same or different.

The hydrogen storage container and second hydrogen storage container may be the same or different.

For the avoidance of doubt, all features relating to the method of the present invention also relate, where appropriate, to the system of the present invention and vice versa.

It should be apparent that each of the second, third, fourth, fifth and sixth aspects of the invention, and each or any of their described variants, may be provided in combination with the first aspect of the invention and each or any of its described variants and/or in combination with any one or more of each other.

### BRIEF DESCRIPTION OF THE DRAWINGS

The invention will now be more particularly described with reference to the following examples and figures, in which;

FIG. 1 is a schematic illustration of a system for increasing hydrogen production from an enclosed bioreactor according to some embodiments herein.

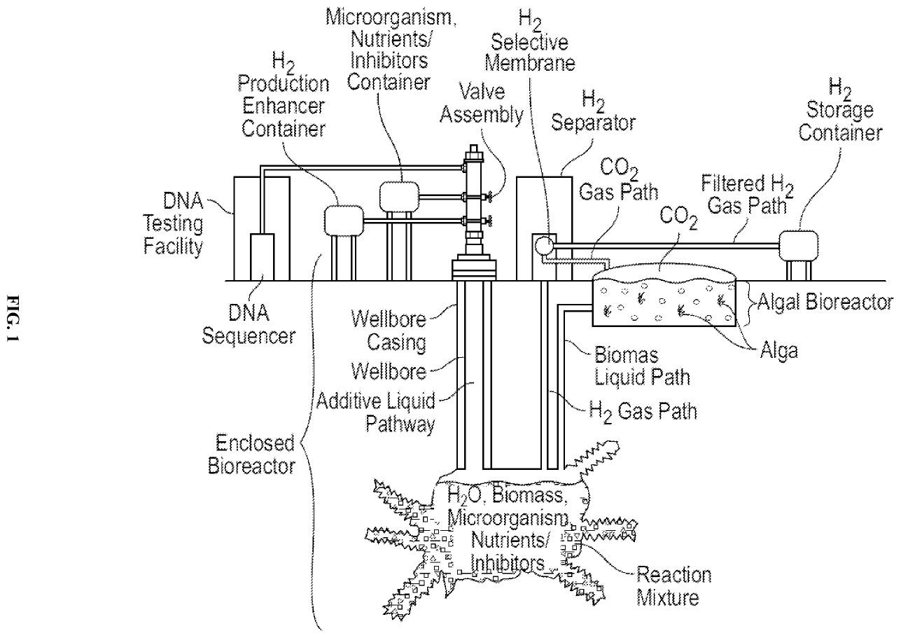

FIG. 2 is a schematic illustration of a system for increasing hydrogen production from an enclosed bioreactor according to some embodiments herein.

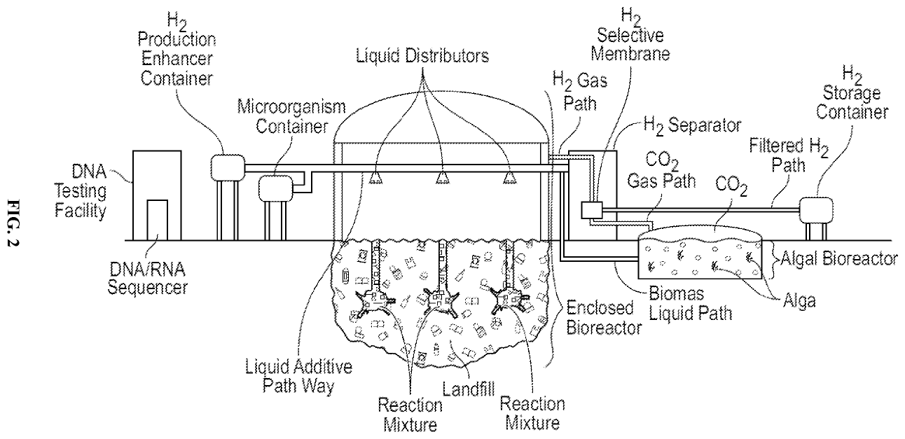

FIG. 3 is a flow chart depicting an embodiment of a method of increasing hydrogen production from an enclosed bioreactor according to some embodiments herein.

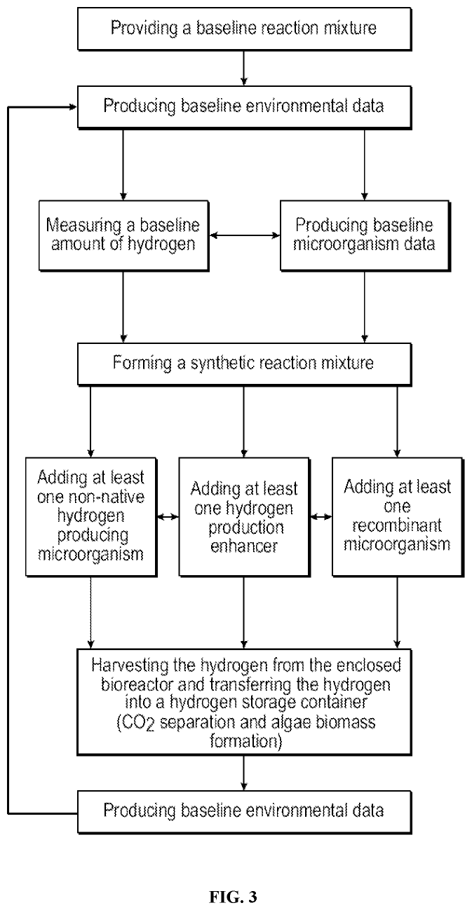

FIG. 4 is a schematic illustration of a system for increasing hydrogen production from an enclosed bioreactor according to some embodiments herein.

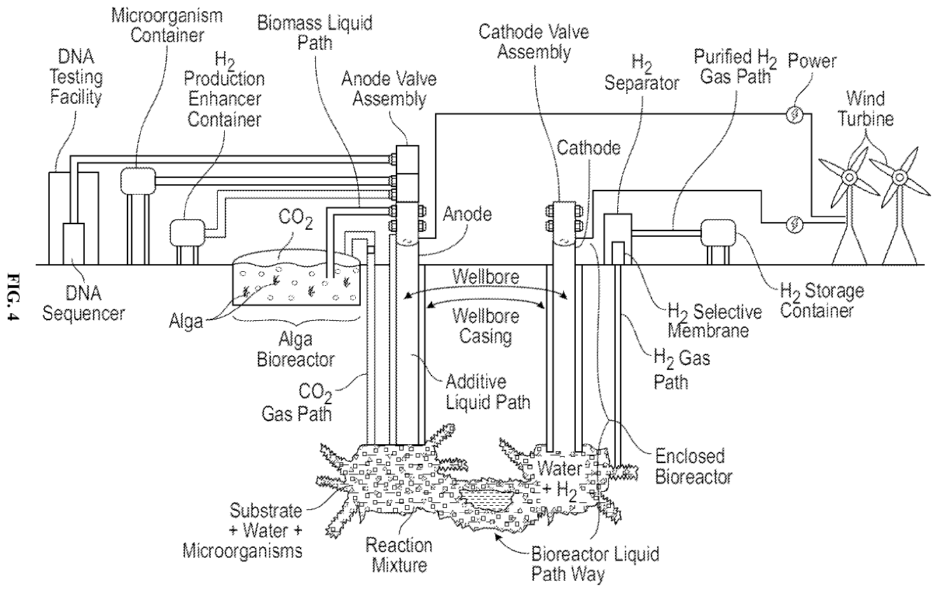

FIG. 5 is a schematic illustration of a system for increasing hydrogen production from an enclosed bioreactor according to some embodiments herein.

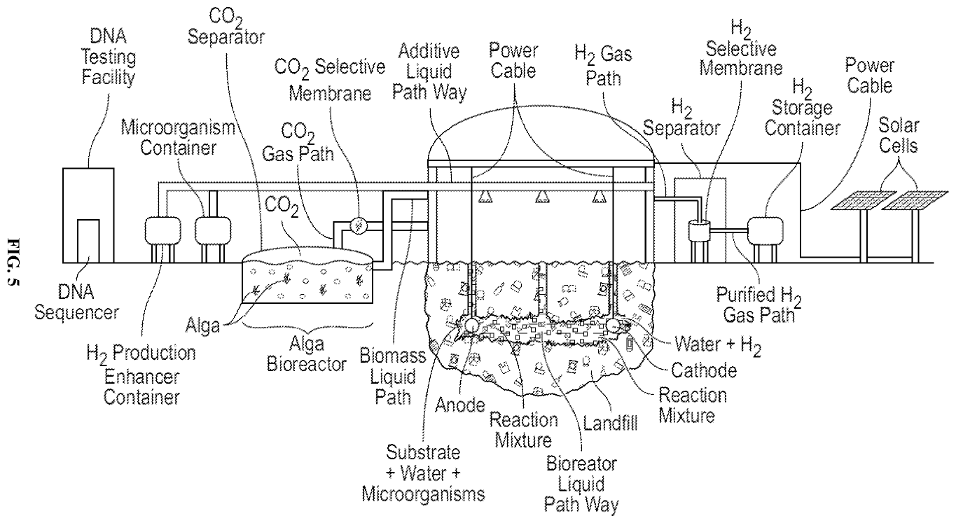

FIG. 6 is a schematic illustration of a system for increasing production of a hydrogen containing liquid from an enclosed bioreactor according to some embodiments herein.

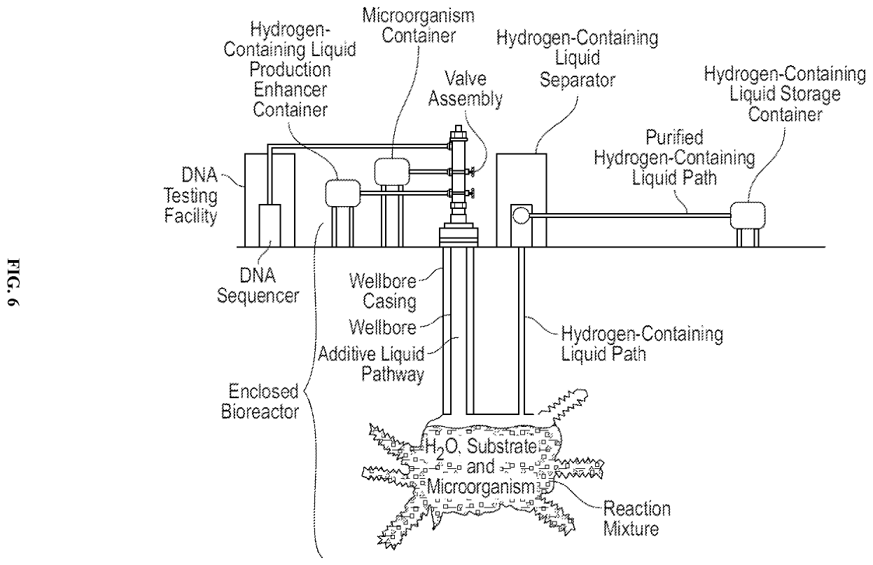

FIG. 7 is a schematic illustration of a system for increasing production of a hydrogen containing liquid from an enclosed bioreactor according to some embodiments herein.

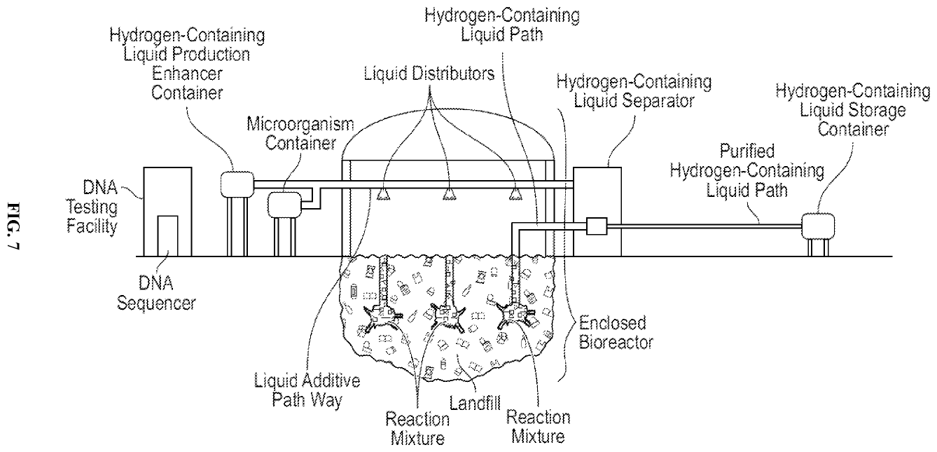

FIG. 8 is a schematic illustration of a system for increasing hydrogen production and hydrocarbon wastewater purification according to some embodiments herein.

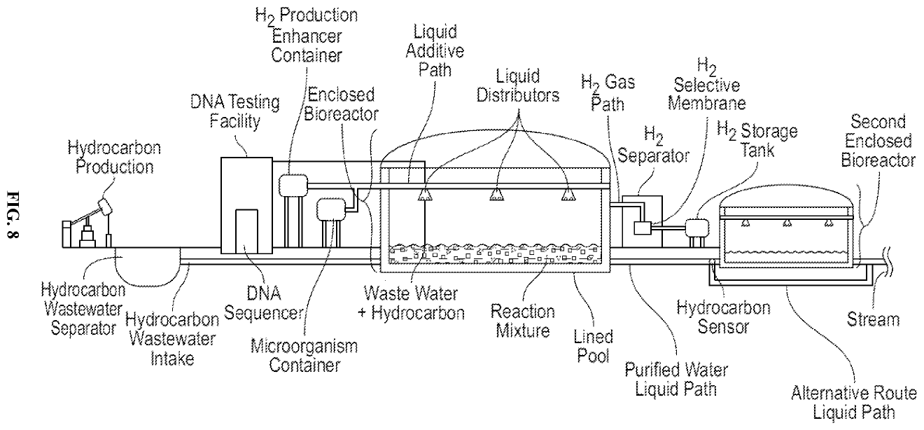

FIG. 9 is a schematic illustration of a system for the microbiological production of hydrogen from a hydrocarbon-rich deposit in accordance with the first aspect of the invention described above, and as exemplified in Example 10 below. It will be apparent to the skilled addressee that recovery of hydrogen from the subterranean deposit may be effected by various means, and that the schematically depicted H₂ separator membrane is merely illustrative.

The foregoing summary, as well as the following detailed description of the embodiments, will be better understood when read in conjunction with the attached drawings. For the purpose of illustration, there are shown in the drawings some embodiments, which may be preferable. It should be understood that the embodiments depicted are not limited to the precise details shown. Unless otherwise noted, the drawings are not to scale.

### DETAILED DESCRIPTION

Unless otherwise noted, all measurements are in standard metric units.

Unless otherwise noted, all instances of the words “a,” “an,” or “the” can refer to one or more than one of the word that they modify.

Unless otherwise noted, the phrase “at least one of” means one or more than one of an object. For example, “at least one of a single walled carbon nanotube, a double walled carbon nanotube, and a triple walled carbon nanotube” means a single walled carbon nanotube, a double walled carbon nanotube, or a triple walled carbon nanotube, or any combination thereof.

Unless otherwise noted, the term “about” refers to ±10% of the non-percentage number that is described, rounded to the nearest whole integer. For example, about 100 mm, would include 90 to 110 mm. Unless otherwise noted, the term “about” refers to ±5% of a percentage number. For example, about 20% would include 15 to 25%. When the term “about” is discussed in terms of a range, then the term refers to the appropriate amount less than the lower limit and more than the upper limit. For example, from about 100 to about 200 mm would include from 90 to 220 mm.

Unless otherwise noted, properties (height, width, length, ratio etc.) as described herein are understood to be averaged measurements.

Unless otherwise noted, the terms “provide”, “provided” or “providing” refer to the supply, production, purchase, manufacture, assembly, formation, selection, configuration, conversion, introduction, addition, or incorporation of any element, amount, component, reagent, quantity, measurement, or analysis of any method or system of any embodiment herein.

Unless otherwise noted, the term “non-native” refers to a microorganism that is not naturally occurring in a particular location, such as a particular subterranean formation.

Unless otherwise noted, the term “recombinant microorganism” refers to a microorganism that does not occur in nature and is the synthetic product of recombinant DNA engineering.

Unless otherwise noted, the term “hydrocarbon” refers to a compound that contains only contains hydrogen and carbon atoms.

Unless otherwise noted, the term “gas path” is interchangeable with the term “gas flow path.” Unless otherwise noted, the term “gas path” refers to an enclosed solid structure or channel that a gas can move or be pumped through. For example, in various embodiments of the systems and methods disclosed herein, a gas path includes one or more pipes and/or tubes connected to or connected through one or more valves or pumps, so long as gas can flow or be pumped continuously through the structure of the gas path.

Unless otherwise noted, the term “liquid path” is interchangeable with the term “liquid flow path.” Unless otherwise noted, the term “liquid path” refers to an enclosed solid structure or channel that a gas can move or be pumped through. For example, in various embodiments of the systems and methods disclosed herein, a gas path includes one or more pipes and/or tubes connected to or connected through one or more valves or pumps, so long as gas can be made to flow or be pumped continuously through the structure of the gas path.

Unless otherwise noted, the term “electrically connected” refers to connecting two or more objects such they can conduct electricity.

Unless otherwise noted, the term “biomass” refers to a product which can contain one or more microorganisms, such as alga, living or dead, colonies of those organisms, and/or the contents of one or more microorganisms, such as enzymes, cytoplasm, nutrients, and the like. An example of a “biomass” can include alga that have been mechanically disrupted.

Unless otherwise noted, the term “enclosed” or “enclosure” refers to a structure that is sealable or resealable, such that when the structure is sealed, the contents of the structure are not free to mix with the open air.

Unless otherwise noted, the term “enclosed bioreactor” refers to a subterranean formation or a landfill enclosure in which a microorganism can be introduced.

Unless otherwise noted, the term “hydrogen-containing liquid” refers to a molecule that contains hydrogen atoms and from 80% to 100% weight of the compound, relative to the total weight of the compound, is a liquid or liquid slurry at standard temperature and pressure.

### EXAMPLES

### Example 1: Initial Set-Up for a Depleted Oil Well

Purchasing or leasing land having a depleted oilwell with a wellbore and a well casing already in place such that the wellbore and well casing extend into a subterranean formation that has been substantially depleted of hydrocarbons. Attaching a valve assembly to the head of the wellbore such that the valves of the valve assembly can control what enters and leaves the wellbore. A suitable valve assembly can be purchased from oil field service companies such as Mogas, Suez Water Technologies, and Halliburton, among others.

Using a bulldozer to dig a pool into the surface within about 100 to 200 meters out of the valve assembly of the depleted oil well. Digging the pool to a depth up about 5 feet any length and width of about 100 meters. The pool would be filled with water and alga of the genera Chlorella or Scenedesmus which can be purchased from UTEX Culture Collection of Algae at the University of Texas at Austin. A series of rods would be extended over the length and width of the pool to form a support structure, and a transparent polyethylene cover would be used to seal the top of the pool, making it substantially airtight. The covered pool would serve as an algal bioreactor.

A free-standing structure would be connected by one or more gas pipes to the algal reactor to form a hydrogen separation building. The hydrogen separation building would be connected to the subterranean formation either directly by drilling a wellbore into the subterranean formation or by one or more pipes connecting to the valve assembly. The freestanding structure would contain a T-junction connecting a gas path from the subterranean formation to a hydrogen selective membrane, where in on one side of the hydrogen selective membrane (the filtered hydrogen side) the hydrogen separator is connected to a hydrogen storage tank by a gas pipe. A suitable hydrogen selective membrane can be hollow microfiber membranes, which can be purchased from Generon located in California, among other suppliers. Alternatively, palladium-based membranes, such as those available from HySep, can be used for hydrogen separation. The other side of the membrane (the carbon dioxide side) would be connected to the algal bioreactor by a gas pipe.

The valve assembly would be connected to two containers, one serving is a microorganism container and one serving as a hydrogen production enhancer container. The valve assembly would further connect to a DNA testing facility wherein the DNA testing facility includes a DNA sequencer and would further connect the valve assembly to the DNA sequencer, such that sequencing could be controlled buy a computer or remotely. A suitable DNA sequencer would include the MinIon nanopore sequencer, which can be commercially purchased from Oxford Nanopore Technologies located in the United Kingdom.

The algal bioreactor would further be connected to the subterranean formation either directly by a well bore and liquid tube or indirectly by connecting the algal bioreactor to the valve assembly.

### Example 2: Initial Set-Up for a Landfill

Purchasing or leasing land having a commercial landfill. Drilling wellbores into the landfill using a commercial drilling rig. Forming liquid distributors by placing one or more pipes over the landfill and drilling holes into the pipes at regular intervals to allow for liquid and slurries to be distributed onto the landfill. Constructing a dome over the landfill to and sealed around the liquid additive pathways, forming a gastight structure that is sealed around the liquid additive pathways. A suitable material for the dome can include polyvinyl chloride, which can be purchased commercially from Membrane Systems Europe located in The Netherlands.

Using a bulldozer to dig a pool into the surface within about 100 to 200 meters out of the valve assembly of the depleted oil well. Digging the pool to a depth up about 5 feet any length and width of about 100 meters. The pool would be filled with water and alga of the genus Chlorella or Scenedesmus which can be purchased from UTEX Culture Collection of Algae at the University of Texas at Austin. A series of rods would be extended over the length and width of the pool to form a support structure, and a transparent polyethylene cover would be used to seal the top of the pool, making it substantially airtight. The covered pool would serve as an algal bioreactor.

A free-standing structure would be connected by one or more gas pipes to the algal reactor to form a hydrogen separation building. The hydrogen separation building would be connected to the landfill by a one gas pipe or tube connected to the liquid additive pathway. The freestanding structure would contain a T-junction connecting a gas path from the landfill dome to a hydrogen selective membrane, where in on one side of the hydrogen selective membrane (the filtered hydrogen side) the hydrogen separator is connected to a hydrogen storage tank by a gas pipe. A suitable hydrogen selective membrane can be hollow microfiber membranes, which can be purchased from Generon located in California, among other suppliers. Alternatively, palladium-based membranes, such as those available from HySep, can be used for hydrogen separation. The other side of the membrane (the carbon dioxide side) would be connected to the algal bioreactor by a gas pipe.

The liquid additive pathway or sprinkler system could be connected to two containers, one serving is a microorganism container and one serving as a hydrogen production enhancer container. The liquid additive pathway or sprinkler system could be separate from or connect to a DNA testing facility. The DNA testing facility would contain a DNA sequencer. A suitable DNA sequencer would include the MinIon nanopore sequencer, which can be commercially purchased from Oxford Nanopore Technologies located in the United Kingdom.

The algal bioreactor would further be connected to the landfill dome by the liquid additive pathway to the algal bioreactor. The liquid additive pathway or sprinkler system could include more than one set of pipes for distributing liquids and slurries. For example, one set of pipes over the landfill might carry a biomass liquid slurry. Another set of pipes could be connected to the microorganism container to distribute the microorganisms over the landfill.

### Example 3: Increasing Hydrogen Production from a Subterranean Formation Having a Low Amount of Hydrogen Producing Microorganisms

Providing the set up according to Example 1 above, with the following changes.

Taking a gas sample of the hydrogen produced by the subterranean information and analyzing the amount of hydrogen in the gas sample using a gas chromatograph with PDHID (Pulse Discharge Helium Ionization Detection) can be purchased from Custom Solutions Group which is located in Houston, Tex. Determining that the hydrogen output is too low.

Taking a liquid sample from the subterranean formation. Performing a bulk DNA extraction by performing the steps detailed in the DNeasy PowerSoil Pro Kit from Qiagen (Hilden, Germany). Quantifying the amount of DNA using real-time PCR and primers that target the 16S rRNA gene. Sequencing the DNA from the samples using a commercially available kit such as the 16S sequencing kit, which is commercially available from Oxford Nanopore Technologies.

Further testing the liquid sample to determine pH, temperature, and level of nutrients present in the liquid sample.

Analyzing the data from the microorganism population and determining that there are microorganisms present in the subterranean formation, but that less than 1% of the microorganisms present produce hydrogen. Adding 1-50 barrels of ˜10E8 cells/mL of a nonnative hydrogen producing microorganism, such as Clostridium spp., which is known to be a hydrogen producing organism and compatible with a pH of 5-8 and temperature of 77-95 F, until the amount is projected to be over 20% of the total microorganisms present. Suitable Clostridium can be purchased from ATCC, which is located in Manassas, Va.

Harvesting an amount of hydrogen by pumping the gasses from the subterranean formation through the hydrogen selective filter into a hydrogen storage tank at a rate of about 0.3 tons/hr to 30 tons/hr, wherein the percentage of hydrogen in the gas sample is increased by at least 10%. Pumping they non-hydrogen gases such as carbon dioxide into the algal bioreactor.

Pumping water, nutrients, and alga from the algal reactor as needed into the subterranean formation to feed the reaction mixture.

Using DNA sequencing to monitor liquid samples about once a month to ensure that the amount of hydrogen producing microbes does not fall below 20% of the total amount of microbes present.

### Example 4: Increasing Hydrogen Production from a Subterranean Formation Having a High Amount of Hydrogen Consuming Microorganisms

Performing the same steps as in Example 3, except the DNA analysis indicates that there are hydrogen producing microorganisms present in an amount of at least 20% of the total amount of microorganisms present, but there is a high amount or percentage of microorganisms such as sulfate reducing microbes or nitrate reducing microbes, which are known to be a microorganism that consumes hydrogen. This hydrogen consumer is decreasing the amount of hydrogen which can be harvested from the subterranean formation. Therefore, instead of adding a native hydrogen producing microorganism, an inhibitor such as sodium nitrate, which is known to inhibit sulfate reducing microbes is pumped into the subterranean formation at about 50 mM concentration.

Taking gas samples from the subterranean formation and adding the inhibitor until the increase in hydrogen percentage relative to the total amount of gases is increased by at least 10%.

### Example 5: Increasing Hydrogen Production from a Subterranean Formation Having a High Temperature and Low pH

Providing this setup according to Example 1 and performing the method according to Example 3 above, except that the DNA analysis of the microbial population and the water testing step indicate that the subterranean formation would be unlikely to support a sustainable population of naturally occurring hydrogen producing microorganisms.

Creating recombinant microorganism by inserting DNA having a sequence, which is known to code for a hydrogen producing protein, into a microorganism, which is known to thrive in environments having the high temperature as well as the low pH. Adding amounts of the recombinant microorganism to the subterranean formation until the total amount of percentage in the population increases above 20% relative to the total population of microorganisms.

Using DNA sequencing to monitor liquid samples from the subterranean formation about once a month to ensure that the amount of recombinant microorganisms does not fall below 20% of the total amount of microbes present.

### Example 6—Applying Potential to Increase Hydrogen Production Using Hydrocarbons in Place as Substrate in the Subterranean Formation

Providing the setup according to Example 1 above.

Electric current would be applied to the reservoir by electrodes placed in water injection wells and production wells. Salt water (recycled produced water) would be injected simultaneously with application of electric current. To reduce the flow of electricity to overlying beds, casing above the electrode would be electrically isolated. Both water and electric current might be transmitted in the well through electrically conductive tubing, so that both the tubing and injected salt water would be utilized as electric conductors. The tubing could be externally insulated, or it could be equipped with non-conductive centralizers and installed with an insulating fluid in the casing-tubing annulus.

Providing the setup according to Example 3, 4, or 5 above.

### Example 7: Applying Potential to Increase Hydrogen Production Using Alternative Organic Mass as Substrate in the Subterranean Formation

Providing the setup according to Example 1 and Example 6 above except there is not enough recalcitrant hydrocarbons left in situ to produce hydrogen to the desired degree.

A biomass consisting of biodegradable waste, paper waste, plant waste, pulp waste, or a combination thereof is pumped into the subterranean formation.

Providing the setup according to Example 3, 4, or 5 above.

### Example 8: Producing Hydrogen Carriers in the Subterranean Formation

Providing the setup according to Example 1 above.

Providing the setup according to Example 3 above except that the DNA analysis is used to determine presence of hydrogen carrier producing microorganisms is less than 1%.

Adding 1-50 barrels of ˜10E8 cells/mL of a non-native hydrogen carrier producing microorganism, such as a recombinant Methanothermobacter which are known to be hydrogen carrier (methanol) producing organisms until the amount is projected to be over 20% of the total microorganisms present. Suitable anaerobic methanotrophs can be isolated from landfills or anaerobic digesters.

Harvesting an amount of hydrogen carriers by pumping the liquids from the subterranean formation into a hydrogen carrier storage tank at a rate of about 0.3 tons/hr to 30 tons/hr, wherein the percentage of hydrogen carrier in the liquid sample is increased by at least 10%. Pumping they non-hydrogen gases such as carbon dioxide into the algal bioreactor. Pumping water, nutrients, and alga from the algal reactor as needed into the subterranean formation to feed the reaction mixture.

Using DNA sequencing to monitor liquid samples about once a month to ensure that the amount of hydrogen producing microbes does not fall below 20% of the total amount of microbes present.

### Example 9: Producing Hydrogen from Oil and Gas Wastewater Treatment Process

Providing the setup according to Example 1 above.

Produced water that has been separated from the total fluids production would be placed in an enclosed bioreactor. Hydrogen would be produced from the enclosed setup providing the setup according to Example 3 above.

### Example 10: Field Well Trial

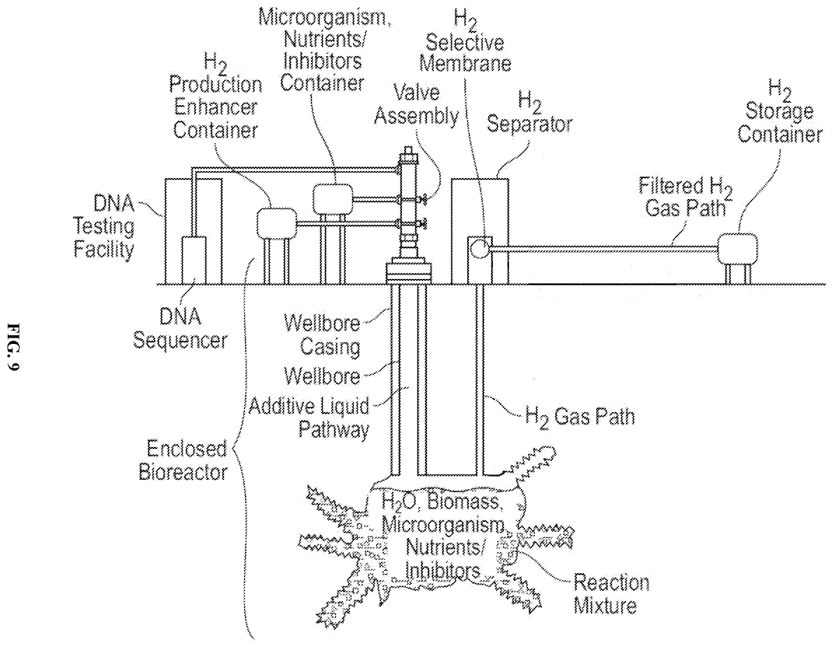

Schematically illustrated (for a single well) in FIG. 9, two low producing oil wells were stimulated in a huff-n-puff application to increase microbial hydrogen production. An oil sample from each well was taken and its indigenous microbiological content determined, with the results set out in Tables 1 and 2 below:

TABLE 1
Well 1 - Indigenous microbial population

| | |
|---|---|
| Halanaerobium praevalens DSM 2228 | 18.1% |
| Acinetobacter johnsonii | 13.4% |
| Desulfohalobium retbaense DSM 5692 | 12.1% |
| Halanaerobium hydrogeniformans | 7.4% |
| Methanohalophilus halophilus | 6.0% |
| Methanohalophilus mahii DSM 5219 | 4.7% |
| Escherichia coli | 2.0% |
| Halobacteroides halobius DSM 5150 | 2.0% |
| Azospirillum thiophilum | 1.3% |
| Keratinibaculum paraultunense | 1.3% |

TABLE 2
Well 2 - Indigenous microbial population

| | |
|---|---|
| Methanohalophilus halophilus | 13.2% |
| Methanohalophilus mahii DSM 5219 | 11.0% |
| Halanaerobium praevalens DSM 2228 | 7.3% |
| Desulfohalobium retbaense DSM 5692 | 6.8% |
| Halanaerobium hydrogeniformans | 3.7% |
| Acinetobacter johnsonii | 3.2% |
| Petrotoga mobilis SJ95 | 3.2% |
| Halothermothrix orenii H 168 | 2.3% |
| Flexistipes sinusarabici DSM 4947 | 2.3% |
| Pelobacter acetylenicus | 1.8% |
| Methanotorris igneus Kol 5 | 1.4% |
| Bacillus mycoides | 1.4% |

In the first well, nutrients were blended as described below in Table 3 into 500 bbls of produced water in a frac tank.

TABLE 3
Nutrient package mixed into the 500 bbls:

| Reagent | [g/L] |
|---|---|
| K₂HPO₄ | 1.044 |
| NH₄Cl | 1.5 |
| Sucrose | 1.41 |
| Yeast extract | 1.5 |
| Tween 80 | 0.081 |

The nutrient mix was injected down the annulus of the well and an additional 500 bbls of produced water was pumped down the annulus on top of the nutrient mixture. In the second well, the same process occurred with the exception that a consortium of microbes capable of producing hydrogen from hydrocarbon fermentation was added to the first 500 bbls of produced water along with the nutrient package.

The consortium was prepared by combining non-native hydrogen producing microorganisms selected to be different from the indigenous microbial populations, and for their capability to digest hydrocarbons to yield hydrogen in preference to methane, in the proportions identified in Table 4:

TABLE 4
Well 2 - Exogenous microbial population

| | |
|---|---|
| Pseudothermotoga elfii | ~20% |
| Pseudothermotoga hypogea | ~20% |
| Thermotoga petrophila | ~20% |
| Petrotoga mobilis | ~20% |
| Caldanaerobacter tengcongensis | ~20% |

The exogenous microbes were maintained in anaerobic liquid culture and nurtured for 2 months under nitrogen (100% N2) at 150 F (65.56 degC), with fresh media inoculated every 3-4 days to provide 100 L kegs for field deployment. The selected media was an ATCC 2114 medium modified for preferential culturing of extremophiles.

Approximately 400 L of microbial culture consisting of approximately 10⁸ cells/mL was added to the 500 bbls.

Following addition of the nutrient package (Well 1) and the nutrient/microbial consortium package (Well 2), the two wells were shut-in for 4 days. After the four-day shut-in period the wells were opened and samples were collected off the gas flow line for analysis with respect to H₂ content on a gas chromatograph, with the results presented in Table 5 below:

TABLE 5
Gas Chromatography characterization of samples:

| | Baseline H2 | After shut-in H2 |
|---|---|---|
| Well | (ppm) | (ppm) |
| 1 (nutrients only) | <112 (LOD) | 1761 |
| 2 (nutrients and microbes) | <112 (LOD) | 13251 |

The gas chromatography was carried out using a standard protocol as follows: 10 milliliter gas samples were extracted from culture bottles using 10 milliliter plastic luer lock syringes. Field gas samples were collected in multi-layer foil gas sampling bags connected via tygon tubing to a sampling valve directly off the of wellhead flow line. Gas samples were injected immediately into the inlet port of an SRI 8610C Gas Chromatograph. The sample was analyzed using a Flame Photometric Detector (FPD), a Flame Ionization Detector (FID), an FID with a large methanizer (FIDM), and a Thermal Conductivity Detector (TCD).

The samples were passed through an 18-inch HayeSep D Packed Column, a 3-foot Molecular Sieve 5A Packed Column, and then into the TCD and FIDM detectors following relay G injection. When relay F was turned on the samples were run through a 6-foot HayeSep D Column and a 60-meter MXT-1 Capillary Column before being analyzed using the FID and FPD. The G relay was turned on at time 0.020 minutes and was turned off at 1.000 minutes, while the F relay was turned on after 4.500 minutes. The initial temperature was set for 50° C. and held for 6 minutes before ramping to 270° C. at a rate of 30° C. per minute. The temperature was held at 270° C. for 6.500 minutes to remove excess sample from the columns.

Any peak areas produced were converted into ppm values using the trend lines of calibration curves derived from standards of various concentrations.

It will be seen from the results in Table 5 that modifying the composition of the well by the introduction into the well of a nutrient package and of consortium of non-native hydrogen producing microorganisms selected positively to diversify the microbiological abundance of hydrogen-producing microorganisms in the well and for the preferential production of hydrogen over methane increased hydrogen production from the well by two orders of magnitude with respect to baseline H₂ production, and by an order of magnitude with respect to introduction of the nutrient package alone.

### Example 11: Microbe Laboratory Data

The consortium of microbes described in Example 10 and capable of producing hydrogen from hydrocarbon fermentation was used to inoculate 6 different synthetic seawater blends in triplicate as described below in Table 6.

TABLE 6
Synthetic seawater blends:

| Brine | Description |
|---|---|
| A | Synthetic seawater |
| B | Synthetic seawater with oil |
| C | Synthetic seawater with nutrients |
| D | Synthetic seawater with nutrients and oil |
| E | Synthetic seawater with enhanced nutrients |
| F | Synthetic seawater with enhanced nutrients and oil |
| G | Synthetic seawater with algae biomass and oil |

Synthetic seawater is a simple reproducible representative of produced water brines. It was produced using NeoMarine aquarium salts by Brightwell Aquatics. The oil used in this example was a sweet west Texas crude blend was used (API 25-35). 4 mL of the oil was used in 100 mL synthetic seawater sample. The nutrient packages employed were as follows in Tables 7, 8 and 9:

TABLE 7
Synthetic seawater with nutrients:

| Reagent | [g/L] |
|---|---|
| Aquarium Salts | 35.40290621 |
| K₂HPO₄ | 0.348 |
| KH₂PO₄ | 0.227 |
| NH₄Cl | 0.5 |
| Wolfes Vitamin solution | 10 mL |
| Reducing agent | 1 |
| Resazurin solution | ~1 mL |
| dH₂O | 989 mL |
| Combine, pH to desired 6.5 +− 0.5), filter sterilize | |

TABLE 8
Synthetic seawater with enhanced nutrient package:

| Reagent | [g/L] |
|---|---|
| Aquarium Salts | 35.40290621 |
| K₂HPO₄ | 0.348 |
| NH₄Cl | 0.5 |
| Glucose | 0.47 |
| Yeast extract | 0.5 |
| Tween 80 | 0.027 |
| Reducing agent | 1 |
| Resazurin solution | ~1 mL |
| dH₂O | 999 mL |
| Combine, pH to desired 6.5 +− 0.5), filter sterilize | |

TABLE 9
Synthetic seawater with algae biomass nutrient package:

| Reagent | [g/L] |
|---|---|
| Aquarium Salts | 35.40290621 |
| K₂HPO₄ | 0 |
| NH₄Cl | 0 |
| Glucose | 0 |
| Yeast extract | 0 |
| Chlorella algae powder | 0.5 |
| Tween 80 | 0.027 |
| Reducing agent | 1 |
| Resazurin solution | ~1 mL |
| dH₂O | 999 mL |

A 100 mL sample of each brine A-E was prepared anaerobically in glass bottles and sealed. Following inoculation, the bottles were incubated at 65 C for 48 hours along with abiotic controls for each brine.

At 48 hours, samples were taken for ATP analysis (microbial enumeration) and gas analysis, the results of which are shown in Table 10.

TABLE 10
ATP Analysis:

| | | Abiotic Control | | Inoculated | |
|---|---|---|---|---|---|
| | | H2 | Microbial | H2 | Microbial |
| | | Concentration | enumeration | Concentration | enumeration |
| Brine | Description | (ppm) | (cells/mL) | (ppm) | (cells/mL) |
| A | Synthetic seawater | 0 | 6.92E+03 | 0 | 1.51E+06 |
| B | Synthetic seawater | 0 | 2.00E+04 | 82.33 | 7.54E+06 |
| | with oil | | | | |
| C | Synthetic seawater | 0 | 1.40E+03 | 119 | 7.55E+06 |
| | with nutrients | | | | |
| D | Synthetic seawater | 0 | 7.67E+03 | 1406.7 | 2.58E+07 |
| | with nutrients and oil | | | | |
| E | Synthetic seawater | 0 | 9.80E+03 | 62.33 | 3.04E+07 |
| | with enhanced | | | | |
| | nutrients | | | | |
| F | Synthetic seawater | 0 | 2.95E+04 | 2735.33 | 2.07E+07 |
| | with enhanced | | | | |
| | nutrients and oil | | | | |
| G | Synthetic sea water | 0 | 1.18E+07 | 2365.5 | 1.01E+08 |
| | with algae biomass | | | | |
| | and oil | | | | |

In sample E, the enhanced nutrient package used causes rapid microbial growth at 24 hours and all of the carbon source is consumed which leads to a lower reading at 48 hours when no oil is present to maintain microbial activity, which rationalizes the lower H₂ concentration observed for this sample relative to comparative sample C.

The test kit used for the determination of ATP was the Luminultra QGO-M which is compliant with ASTM Standard E2694 for the measurement of ATP in Metalworking Fluids and D7687 for the measurement of ATP in fuels, fuel/water mixtures and fuel-associated water.

1. A process for the microbiological production of hydrogen from a hydrocarbon-rich deposit, said process comprising the step of modifying the composition of the deposit by the introduction into the deposit of at least one non-native hydrogen producing microorganism selected positively to diversify the microbiological abundance of hydrogen-producing microorganisms in the deposit and for the preferential production of hydrogen over methane.

2. The process according to claim 1, wherein the non-native hydrogen producing microorganism is:
a. a microorganism not naturally present in the hydrocarbon-rich deposit; and/or
b. of a strain of microorganisms not naturally present in the hydrocarbon-rich deposit; and/or
c. of a species of microorganisms not naturally present in the hydrocarbon-rich deposit; and/or
d. of a genus of microorganisms not naturally present in the hydrocarbon-rich deposit; and/or
e. a microorganism naturally present in the hydrocarbon-rich deposit but genetically modified to increase (relative to the naturally present microorganism) its propensity for hydrogen production by the metabolization by that microorganism of one or more hydrocarbons contained within the deposit.

3. The process according to claim 1, wherein the at least one non-native hydrogen producing microorganism is one of a plurality of different non-native hydrogen producing microorganisms, strains of microorganisms, species of microorganisms, genera of microorganisms and/or naturally occurring but genetically modified organisms introduced into the deposit.

4. The process according to claim 3, wherein the plurality is greater than two, greater than three, greater then four and/or greater than five.

5. The process according to claim 1, wherein the non-native hydrogen producing microorganism has a propensity to metabolize one or more hydrocarbons contained within the deposit to molecular hydrogen in preference to methane such that the yield of production of molecular hydrogen from the metabolization is higher than the yield of production of methane by at least 1%, by at least 10%, by at least 100% and/or by at least 1000%.

6. The process according to claim 1, wherein the non-native hydrogen producing microorganism is introduced into the deposit and accompanied during, after or upon its introduction by at least one nutrient selected to promote the growth of said microorganism and introduced into the deposit for that purpose.

7. The process according to claim 6, wherein the at least one nutrient is selected preferentially to promote the growth of the said microorganism in preference to at least one, to at least some or to all of any native microorganisms in the deposit.

8. The process according to claim 6, wherein the nutrient comprises one or more of:
a. one or more salts selected from:
i. phosphates; and/or
ii. halides; and/or
iii. nitrates/ammonium salts/nitrogenous salts
b. one or more carbohydrates selected from:
i. sugars; and/or
ii. starches; and/or
c. one or more vitamins;
d. complex nutrients, optionally selected from yeast extracts, corn steep liquor, biomass, bacterial and/or algal biomass.

9. The process according to claim 1, wherein the hydrogen producing microorganism is introduced into the deposit and accompanied during, after or upon its introduction by at least one pH regulator selected to regulate the pH environment in which the microorganism resides in the deposit and introduced into the deposit for that purpose.

10. The process according to claim 9, where in the pH regulator is selected to regulate the pH of the hydrogen producing microorganism environment in the deposit to a pH within the range of from about 5 to about 9, from about 6 to about 8 and/or from about 6 to about 7.

11. The process according to claim 1, wherein the hydrocarbon-rich deposit is a liquid hydrocarbon-rich deposit.

12. The process according to claim 1, wherein the at least one non-native hydrogen producing microorganism has a genus of Syntrophobacter, Syntrophus, Syntrophomonas, Thermoanaerobacter, Thermotoga, Pseudothermotoga, Thermoanaerobacterium, Fervidobacterium, Thermosipho, Haloanaerobium, Acetoanaerobium, Anaerobaculum, Geotoga, Petrotoga, Thermococcus, Pyrococcus, Clostridium, Enterobacter, Klebsiella, Ethanoligenens, Pantoea, Escherichia, Bacillus, Caldicellulosiruptor, Pelobacter, Caldanaerobacter, Marinitoga, Oceanotoga, Defluviitoga, Kosmotoga, or a combination or mixture thereof.

13. The process according to claim 12, wherein the non-native hydrogen producing microorganism or the recombinant microorganism expresses at least one protein selected from hydrogenases, dehydrogenases, hydroxylases, carboxylases, esterases, hydratases and acetyltransferases having an amino acid sequence at least 95% identical to a sequence expressed by an upregulated or downregulated gene selected from mth (EC 1.12.98.2), mrt, hycA (ID: 45797123), fdhF (ID: 66346687), fhlA (ID: 947181), ldhA (ID: 946315), nuoB (ID: 65303631), hybO (ID: 945902), fdhl, narP, ppk or Pepc by expressing a non-native protein expressing nucleotide sequence, wherein an amount of hydrogen produced or protein produced by the non-native hydrogen producing microorganism or the recombinant microorganism is greater than that produced relative to a control microorganism lacking the non-native protein expressing nucleotide sequence.

14. The process according to claim 1, wherein the environment of the hydrocarbon-rich deposit and the introduced hydrogen producing microorganism constitutes an enclosed bioreactor, being a bioreactor subterranean formation, a bioreactor landfill enclosure, or a combination thereof.

15. The process according to claim 14 comprising:
a. providing a baseline reaction mixture in the enclosed bioreactor, wherein the baseline reaction mixture includes a hydrocarbon having up to 120 carbon atoms, water, and a baseline amount of at least one microorganism; producing baseline microorganism data on an identity and a baseline percentage of the at least one microorganism, relative to a baseline total percentage of microorganisms in the baseline reaction mixture, by performing DNA and/or RNA sequencing of a baseline microorganism sample from the baseline reaction mixture; measuring a baseline amount of hydrogen in a baseline gas sample of gasses collected from the enclosed bioreactor; increasing hydrogen production from the enclosed bioreactor by forming a synthetic reaction mixture, and harvesting the hydrogen from the enclosed bioreactor at a hydrogen harvesting rate by separating the hydrogen from other gasses and transferring the hydrogen into a hydrogen storage container; and/or
b. providing at least one anode and at least one cathode connected to an interior of the enclosed bioreactor, wherein the enclosed bioreactor is a subterranean formation, an enclosed landfill, or a combination thereof, and the at least one anode and the at least one cathode are connected through the enclosed bioreactor by at least one bioreactor liquid pathway; providing a baseline reaction mixture in the enclosed bioreactor, wherein the baseline reaction mixture includes an organic substrate, water, and a baseline amount of at least one microorganism; measuring a baseline amount of hydrogen in a baseline gas sample of gasses collected from the enclosed bioreactor; increasing hydrogen production from the enclosed bioreactor from the baseline amount of hydrogen to a production amount of hydrogen by applying a potential between the at least one anode and the at least one cathode; and harvesting the hydrogen from the enclosed bioreactor at a hydrogen harvesting rate by separating the hydrogen from other gasses and transferring the hydrogen into a hydrogen storage container, wherein the production amount of hydrogen is at least 20% greater than the baseline amount of hydrogen; and/or
c. providing a baseline reaction mixture in the enclosed bioreactor, wherein the baseline reaction mixture includes a substrate, water, and a baseline amount of at least one microorganism, wherein the substrate includes a nitrogen source, an unsaturated hydrocarbon having from 2 to 120 carbon atoms, methane, hydrogen, or a combination thereof, wherein the hydrogen-containing liquid includes ammonia, ammonium, methanol, a saturated hydrocarbon having from 2 to 120 carbon atoms, or a combination thereof; producing baseline microorganism data on an identity and a baseline percentage of the at least one microorganism, relative to a baseline total percentage of microorganisms in the baseline reaction mixture, by performing DNA and/or RNA sequencing of a baseline microorganism sample from the baseline reaction mixture; measuring a baseline amount of hydrogen-containing liquid in a baseline sample collected from the enclosed bioreactor; increasing production of the hydrogen-containing liquid from the enclosed bioreactor by forming a synthetic reaction mixture, and harvesting the hydrogen-containing liquid from the enclosed bioreactor at a hydrogen-containing liquid harvesting rate by separating the hydrogen-containing liquid from solids and other liquids by transferring the hydrogen-containing liquid into a hydrogen-containing liquid storage container; and/or
d. providing hydrocarbon wastewater from a hydrocarbon producing site; forming a baseline reaction mixture by transferring the hydrocarbon wastewater into an enclosed bioreactor, wherein the baseline reaction mixture includes the hydrocarbon wastewater and a baseline amount of at least one microorganism; producing baseline microorganism data on an identity and a baseline percentage of the at least one microorganism, relative to a baseline total percentage of microorganisms in the baseline reaction mixture, by performing DNA and/or RNA sequencing of a baseline microorganism sample from the baseline reaction mixture; measuring a baseline amount of hydrogen in a baseline gas sample of gasses collected from the enclosed bioreactor; measuring a baseline amount of hydrocarbons in a baseline liquid sample of a liquid collected from the enclosed bioreactor; producing hydrogen and forming purified water from the hydrocarbon wastewater by forming a synthetic reaction mixture in the enclosed bioreactor, harvesting the hydrogen from the enclosed bioreactor at a hydrogen harvesting rate by separating the hydrogen from other gasses and transferring the hydrogen into a hydrogen storage container, and gathering the purified water from the enclosed bioreactor by transferring the purified water from the enclosed bioreactor to a purified water liquid path at a purified water rate, optionally of from about 10 L/hr to about 10,000 L/hr; and/or

16. The process according to claim 14 for increasing hydrogen production from the enclosed bioreactor comprising:
a. providing a baseline reaction mixture in the enclosed bioreactor, wherein the baseline reaction mixture includes a hydrocarbon having up to 120 carbon atoms, water, and a baseline amount of at least one microorganism;
b. producing baseline microorganism data on an identity and a baseline percentage of the at least one microorganism, relative to a baseline total percentage of microorganisms in the baseline reaction mixture, by performing DNA and/or RNA sequencing of a baseline microorganism sample from the baseline reaction mixture;
c. measuring a baseline amount of hydrogen in a baseline gas sample of gasses collected from the enclosed bioreactor;
d. increasing hydrogen production from the enclosed bioreactor by forming a synthetic reaction mixture, and
e. harvesting the hydrogen from the enclosed bioreactor at a hydrogen harvesting rate by separating the hydrogen from other gasses and transferring the hydrogen into a hydrogen storage container;
f. forming the synthetic reaction mixture by:
i. adding at least one non-native hydrogen producing microorganism until a percentage of the non-native hydrogen producing microorganism in the synthetic reaction mixture is at least 20% of a total amount of microorganisms in the synthetic reaction mixture; or
ii. adding at least one hydrogen production enhancer to the baseline reaction mixture until a post-baseline amount of hydrogen in a post-baseline gas sample of gasses collected from the enclosed bioreactor is at least 10% higher than the baseline amount of hydrogen; or
iii. adding at least one recombinant microorganism to the baseline reaction mixture until a percentage of the at least one recombinant microorganism in the synthetic reaction mixture is at least 20% of a total amount of microorganisms in the reaction mixture, or
iv. a combination thereof.

17. The process according to claim 16 wherein:
a. the hydrogen production rate of the enclosed bioreactor is from about 0.1 L/hr to about 10⁶ L/hr; and/or
b. the enclosed bioreactor is a subterranean formation comprising a natural formation, non-natural formation, a hydrocarbon-bearing formation, a natural gas-bearing formation, a methane-bearing formation, a depleted hydrocarbon formation, a depleted natural gas-bearing formation, a wellbore, or a combination thereof; and/or
c. the enclosed bioreactor is a landfill enclosure comprising a landfill that is enclosed by a building material, wherein the building material includes at least one of a brick, a cement, a plastic, a non-natural rubber, a geomembrane of any kind, concrete, steel, a glass, or a combination thereof; and/or
d. the hydrogen production enhancer is a biocidal inhibitor (optionally glutaraldehyde, a quaternary ammonium compound, formaldehyde, a formaldehyde releaser such as 3,3′-methylenebis[5-methyloxazolidine], dibromonitrilopropionamide, tetrakis hydroxymethyl phosphonium sulfate, chlorine dioxide, peracetic acid, tributyl tetradecyl phosphonium chloride, methylisothiazolinone, chloromethylisothiazolinone, sodium hypochlorite, dazomet, dimethyloxazolidine, trimethyloxazolidine, N-Bromosuccinimide, Bronopol, or 2-propenal, or a mixture thereof), a methanogenesis inhibitor (optionally bromethane sulfonic acid, an Aminobenzoic acid, 2-bromoethanesulfonate, 2-chloroethanesulfonate, 2-mercaptoethanesulfonate, lumazine, a fluoroacetate, nitroethane, or 2-nitropropanol, or a mixture thereof), a sulfate reduction inhibitor (optionally a molybdate salt, a nitrate salt, a nitrite salt, a chlorate salt, or a perchlorate salt or a mixture thereof), a nitrate reduction inhibitor (optionally sodium chlorate, a chlorate salt, or a perchlorate salt, or a mixture thereof), an iron reduction inhibitor, or a combination thereof.

18. The process according to claim 16, further comprising:
a. producing carbon dioxide from the enclosed bioreactor at a carbon dioxide producing rate,
b. separating the carbon dioxide from other gasses by filtering the carbon dioxide through a carbon dioxide-selective membrane filter; and
i. pumping the carbon dioxide into the enclosed bioreactor at a replenishment rate or to a different enclosed bioreactor at an injection rate; and/or
ii. forming an algal biomass by reacting the carbon dioxide with an algae reaction mixture in an algal bioreactor, and pumping the algal biomass into the reaction mixture of the enclosed bioreactor or a different enclosed bioreactor.

19. The process according to claim 16, wherein forming the synthetic reaction mixture includes:
a. adding at least one non-native hydrogen producing microorganism until a percentage of the non-native hydrogen producing microorganism in the synthetic reaction mixture is at least 20% of a total amount of microorganisms in the synthetic reaction mixture; and/or
b. adding at least one hydrogen production enhancer to the baseline reaction mixture until a post-baseline amount of hydrogen in a post-baseline gas sample of gasses collected from the enclosed bioreactor is at least 10% higher than the baseline amount of hydrogen; and/or
c. adding at least one recombinant microorganism to the baseline reaction mixture until a percentage of the at least one recombinant microorganism in the synthetic reaction mixture is at least 20% of a total amount of microorganisms in the reaction mixture.
